(19) United States
(12) Patent Application Publication
(10) Pub. No.: US 2023/0060706 A1
(43) Pub. Date: Mar. 2, 2023

(54) Electrostatic Sprayer

(71) Applicant: Shephia Waldon, Stafford, VA (US)

(72) Inventor: Shephia Waldon, Stafford, VA (US)

(73) Assignee: Supereon, LLC, Fredericksburg, VA (US)

(21) Appl. No.: 17/464,475

(22) Filed: Sep. 1, 2021

(51) Int. Cl. B05B 5/025; B05B 5/053; B05B 5/16

(52) U.S. Cl. CPC B05B 5/0255; B05B 5/0533; B05B 5/0531; B05B 5/1691

(57) ABSTRACT

The present invention is directed to an electrostatic sprayer for applying disinfectants, agricultural production, pesticides, and other chemicals and liquid solutions. The electrostatic sprayer may be used for indoor and outdoor applications. The electrostatic sprayer provides a solution tank for holding a treatment solution, a base for holding and protecting the mechanical and electrical components, including a power supply, solution pump, and electrostatic generator, of the electrostatic sprayer, a supply hose, and a spray wand. The solution tank further includes input and exit filters to remove particles that may damage the solution pump of clog the spray wand. The spray wand includes spray nozzles with backflow valves and metering disks to control the flow of the treatment solution and close the valve when the treatment solution is not sufficiently pressurized to pass through the backflow valve to prevent leak when not in use. An electrostatic switch is also provided for selectively powering on the electrostatic generator. The electrostatic generator provides a positive charge to an electrode in the solution tank and a negative charge to a grounding strip in the handle of the spray wand providing a negative pathway through Earth to the objects being treated enhancing the electrical attraction of the treatment solution to those items. A grounding strap is also provided to be worn by the user to reduce the resistance in the path between the grounding strip and Earth by bypassing some of the user's clothing and protective gear that may provide resistance to the path.

### CROSS REFERENCE TO RELATED APPLICATIONS

This application claims benefit of U.S. Design patent application 29/748,759 entitled “Electrostatic Sprayer” and filed Sep. 1, 2020; U.S. Design patent application 29/750,237 entitled “Electrostatic Sprayer” and filed Sep. 11, 2020; and U.S. Design patent application 29/750,258 entitled “Electrostatic Sprayer” and filed Sep. 11, 2020, which are herby incorporated by reference.

### BACKGROUND OF THE INVENTION

### 1. Field of the Invention

The present invention relates to an electrostatic sprayer for both indoor and outdoor applications for applying disinfectants, agricultural products, pesticides, and other chemical and liquid products when the electrostatic feature is selectively enabled or disabled in order to disable the electrostatic charging of a treatment solution when spraying electrical charge sensitive objects.

### 2. Discussion of the Related Art

Foggers and misters are typically used to create a dry fog or wet mist and have been used to apply fluids to a variety of objects and surfaces, including for disinfecting the air and surrounding surfaces, as well as applying agricultural fertilizers, chemicals, and pesticides. Electrostatic sprayers with an electrostatic charging system enhance the functionality of foggers and misters by including an electrode that induces an electrical charge to the solution, making it actively attracted to surfaces with a naturally negative charge allowing the solution to more fully encapsulate objects for complete coverage.

While foggers and misters are common they typically provide spotty coverage and can be significantly affected by ventilation and air circulation around the space and objects being treated. Additionally, the fog or mist may also stay airborne for a prolonged period of time making the treated area a potential health hazard where the fog or mist posses an inhalation danger for those in the treated area after treatment has taken place.

Electrostatic sprayers have improved on foggers and misters by providing an electrostatic positive charge to the solution wherein the sprayed solution is electrically attracted to the surrounding surfaces, which has a naturally occurring negative charge, allowing for a more consistent coverage. As the treatment is spread more evening it allows the fluid to dry more quickly.

While electrostatic sprayers are becoming more common, there are instances where objects being treated are electrically sensitive and should not be sprayed with an electrically charged treatment solution, for example, areas where flammable fuels or other liquids are located or where there are electronic devices that are sensitive to static buildup. Accordingly, it may be necessary to carry a mister and an electrostatic sprayer to be able to treat a particular location necessitating the need to carry treatment solution for both devices, as well.

The need to transfer treatment solution to the solution tank of an electrostatic sprayer also creates the likelihood that dust, dirt, or other particles will be transferred into the solution tank. Such items may interfere with a solution pump or create buildup within the delivery hoses, wands, or nozzles of the electrostatic sprayer.

Electrostatic sprayers typically use a power source that is designed for the particular sprayer with no ability to change the physically size of the power source even if the electrical specifications of the differently sized power supply could work for the device.

Electrostatic sprayers also tend to leak once the solution pump has been turned off as the nozzles continue to allow fluid to flow even when the fluid is not pressurized. When the fluid leaks from a nozzle it may create a siphoning reaction, which then continues to draw treatment solution from the solution tank draining the tank and possibly creating large spills of concentrated treatment solution.

Various attempts have been made to enhance the electrostatic charge difference between the treatment solution and the objects being treated. Several have attempted to use the user's body to create continuity to Earth but these efforts have been unable to deal with the wide variety of charge carrying capabilities of the human body and the wide range of clothing and protective gear used. Accordingly, both the user's body and the clothing, including gloves and shoes, can limit the ability to transfer the charge through the user to Earth.

Furthermore, various components of electrostatic sprayers tend to corrode with the use of most treatment solutions requiring disposal or repair of the electrostatic sprayer on a regular basis.

### SUMMARY OF THE INVENTION

Accordingly, the present invention is directed to an electrostatic sprayer that provides a more efficient electrostatic process by creating a solution with a positive electrostatic charge generated by an electrostatic generator, as well a direct negative connection generated by the electrostatic generator and the objects being treated. The negative connection path is created by connecting the negative output of the electrostatic generator to a grounding strip within the spray wand handle of the electrostatic sprayer. A grounding strap is also applied to the user's footwear with a heal strap, securing straps, and a grounding lace that is then placed within the user's shoe or sock providing a more direct connection between the user' body and the Earth.

As the grounding strip is in contact with the user of the electrostatic sprayer and the electrostatic strap is in contact with the user and Earth, the negative connection is passed through the user and the ground where it enhances the negative charge of the objects being treated.

According to various embodiments, the electrostatic sprayer of the present invention includes a tank, a base, a spray wand, and backpack straps. The tank holds the treatment solution and has a fill opening for filling the tank with the treatment solution. According to an embodiment of the present invention a first tank filter is removeably disposed within the fill opening to ensure any debris in the treatment solution supply is filtered out before it enters the solution tank. The fill opening is then capped with a lid to block any spillage of the treatment solution.

The tank also has an exit port for supplying the treatment solution to a solution pump located within the base. According to a further embodiment of the present invention, a second tank filter for filtration of smaller particles those blocked by the first filter is located within the tank at the exit port as an additional layer of protection against any debris that may have found its way into the tank; thus, protecting the inner workings of the solution pump.

The tank is connected to the base section, which contains the electronic components of the present invention, including a power supply, the solution pump, and the electrostatic generator. To electrostatically charge the solution, the electrostatic generator is electrically connected to a positive electrostatic electrode that is positioned within the tank. The electrode of the present invention also resists corrosion; thus limiting the need to replace the electrode on a regular basis.

Accordingly, the electrostatic generator provides a positive electrostatic charge to the solution inside the tank via the electrode. The electrostatic generator is also electrically connected to a grounding strip in the handle of the spray wand. The grounding strip is able to pass the negative charge through the user and when used with the grounding strap provides an increased negative charge to the items being treated; thus, providing direct connection between the electrostatic generator and the items being treated.

The pump is interconnected between the tank and the spray wand and pressurizes the solution for application. According to various embodiments of the present invention a variety of nozzles may interconnect with the spray wand.

The nozzles of the present invention further include a backflow valve to reduce the amount of treatment solution the exits the nozzles after the supply pump is turned off. When the treatment solution Is not pressurized the backflow valves in the nozzles close to stop further treatment solution from exiting the nozzle thus limiting leaking and the possibility of a siphoning action being created and creating a leak or emptying the tank when the electrostatic sprayer is not in use.

The electrostatic sprayer of the present invention also provides a customizable power supply bay to allow for a variety of power supply sizes to be used in the same device. Various moveable walls are included within the power supply bay that can be relocated to accommodate a variety of power supply sizes.

Accordingly, it is an object of the electrostatic sprayer according to the present invention to provide a more effective application of the treatment solution to surrounding surfaces by providing both a positive and negative charge for use in the electrostatic spraying process.

It is a further object of the present invention to provide an electrostatic sprayer where the electrostatic generator may be turned off while the solution pump continues to pressurize treatment solution. allowing the electrostatic sprayer to continue to be used as a sprayer when an electrostatic charge could damage items in the treatment area.

It is also an object of the present invention to provide an electrostatic sprayer with grounding elements at the user's hand and foot to more efficiently pass a negative electrostatic charge through the user's body to the treated surfaces.

It is further an object of the present invention to provide an electrostatic sprayer with one or more filters within the tank to filter debris that may attempt to enter or has already entered the treatment tank.

It is another object of the present invention to provide an electrostatic sprayer with nozzles with backflow valves to present discharge and leakage of the treatment solution when the solution pump is turned off.

It is a further object of the present invention to provide a customizable power supply compartment to allow for the changing of power supply sizes and capacities.

It is yet a further embodiment of the present invention to provide an electrostatic sprayer that is wearable as a backpack to provide enhanced portability.

These and other features and advantages will be set forth in the description that follows, and in part will be apparent from the description, or may be learned by practice of the invention. The objectives and other advantages of the present invention will be realized and attained by the structure particularly pointed out in the written description and claims hereof, as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide further understanding of the invention and are incorporated in and constitute a part of this specification, illustrate embodiments of the invention and together with the description serve to explain the principles of the invention. In the drawings:

FIG. 1 shows a perspective view of an electrostatic sprayer in accordance with an embodiment of the present invention;

FIG. 2a shows a perspective view of an electrostatic sprayer with a spray wand with two nozzles in accordance with an embodiment of the present invention;

FIG. 2b shows a perspective view of an electrostatic sprayer with a spray wand with four nozzles in accordance with an embodiment of the present invention;

FIG. 3 shows a perspective view of another embodiment of the electrostatic sprayer in accordance with the present invention;

FIG. 4 shows perspective view of an additional embodiment of the electrostatic sprayer according to an embodiment of the present invention;

FIG. 5 shows a rear perspective view of an electrostatic sprayer in accordance with an embodiment of the present invention;

FIG. 6a shows a right-side view of an electrostatic sprayer according to an embodiment of the present invention;

FIG. 6b shows a left-side view of an electrostatic sprayer according to an embodiment of the present invention;

FIG. 7 shows a front view of an electrostatic sprayer in accordance with an embodiment of the present invention;

FIG. 8 provides a bisected front view of an electrostatic sprayer according to an embodiment of the present invention;

FIG. 9 shows a bottom view of an electrostatic sprayer according to an embodiment of the present invention;

FIG. 10 shows a bottom view with the base plate removed according to an embodiment of the present invention;

FIG. 11 shows a wiring diagram of an electrostatic sprayer according to an embodiment of the present invention;

FIG. 12 shows a detailed view of the spray wand handle in accordance with an embodiment of the present invention;

FIG. 13 shows a grounding strap for use with the electrostatic sprayer according to embodiments of the present invention.

FIG. 14 shows a bisected view of a spray nozzle according to an embodiment of the present invention;

FIG. 15a provides a perspective top view of the metering disks of a spray nozzle in accordance with an embodiment of the present invention;

FIG. 15b provides a perspective bottom view of the metering disks of a spray nozzle in accordance with an embodiment of the present invention; and

FIG. 16 provides a bisected, detailed view of the backflow valve in accordance with an embodiment of the present invention.

### DETAILED DESCRIPTION OF THE PREFERRED EMBODIMENTS

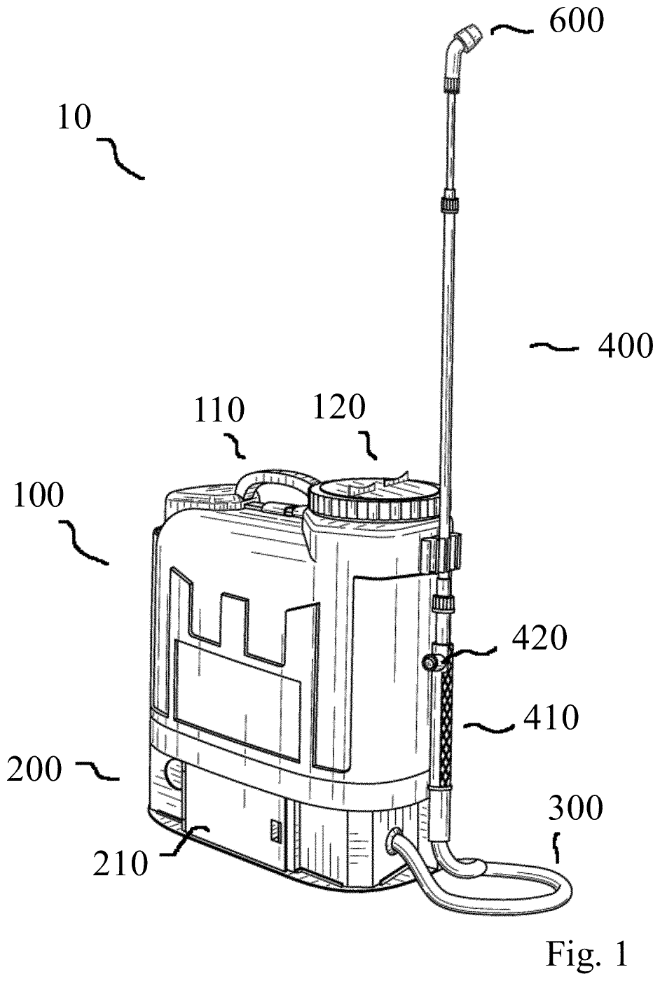

Referring now to the drawings in more detail, FIG. 1 shows a perspective view of an electrostatic sprayer 10 in accordance with an embodiment of the present invention. According to the embodiment shown in FIG. 1, the electrostatic sprayer 10 includes a solution tank 100 for holding treatment solution to be sprayed, a base 200 for housing various mechanical and electrical components, a supply hose 300 for delivering pressurized treatment solution, a spray wand 400 for spraying the pressurized treatment solution, and one or more spray nozzles 600.

According to various embodiments of the present invention, the solution tank 100 as shown in FIG. 1 includes a handle 110 for ease of transport and a tank lid 120, which can be removed to fill the solution tank 100 with the treatment solution for spraying on the treated area. While in use, the tank lid 120 prevents spillage of the treatment solution by sealing the solution tank 120.

According to various embodiments, solution tank 100 and base 200 may be constructed of a variety of materials, including plastics, composites, or metals. Additional materials for constructing the solution tank 100 and base 200 will be apparent to one skilled in the art.

As shown in FIG. 1, according to one embodiment, the solution tank 100 is interconnected with base 200. Solution tank 100 and base 200 may be interconnected with a variety of methods, including, but not limited to heat fusion, gluing, press fit, or screwing together. Other methods of interconnecting the solution tank 100 with the base 200 will be apparent to one skilled in the art. Further embodiments may also provide configurations wherein the solution tank 100 and base 200 are separate elements interconnected with an additional hose to transfer the treatment solution from the solution tank 100 to the base 200.

FIG. 1 further provides a view of an access door 210 in base 200, which is used for accessing a power supply compartment for removing and replacing a power supply for the electrostatic sprayer 10. According to various embodiments of the present invention, access door 210 may be sized to allow a variety of different sized power supplies, which can allow for a lighter electrostatic sprayer when a smaller power supply is needed, or allow for a larger sized power supply when there is a need for expanded power capabilities.

The electrostatic sprayer 10, as shown in FIG. 1, further includes the supply hose 300 that is interconnected between a solution pump within the base 200 and the spray wand 400. The supply hose 300 comprises an internal hose and an outer conduit for protecting the internal hose and provides pressurized solution from the solution pump to the spray wand 400 for spraying the application of the treatment solution through the spray nozzle 600.

The flow of the pressurized treatment solution is controlled with the wand switch 420 located in the wand handle 410. Electrical wiring from the wand handle 410 to the base 200 travels through the supply hose 300 between the internal hose and the outer conduit. When the wand switch 420 is turned on the solution pump drives treatment solution from the solution tank 100 through the supply hose 300 to the wand 400 and through spray nozzle 600. When the wand switch 420 is turned off the solution pump stops pressurizing the treatment solution and once the pressure is reduced the nozzle 600 stops any remaining treatment solution from flowing.

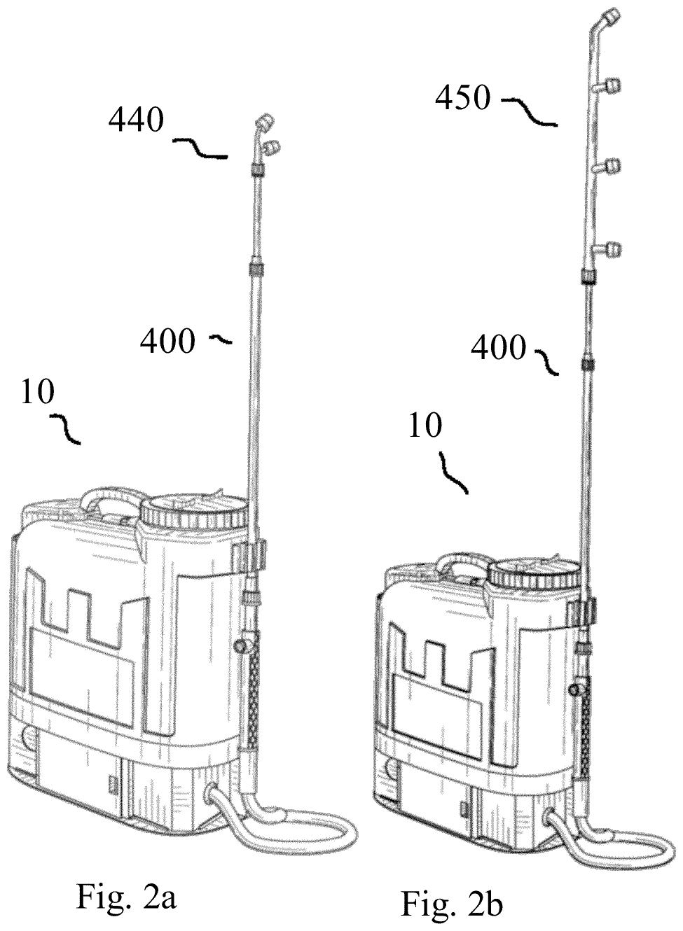

FIGS. 2a and 2b provide additional embodiments of the spray wand 400 wherein in the spray wand 400 of FIG. 2a further includes a double nozzle configuration 440 and the spray wand 400 of FIG. 2b shows spray wand 400 with a quad-nozzle configuration 450. Further embodiments of the present invention may include additional nozzle configurations an will be apparent to one skilled in the art of electrostatic sprayers.

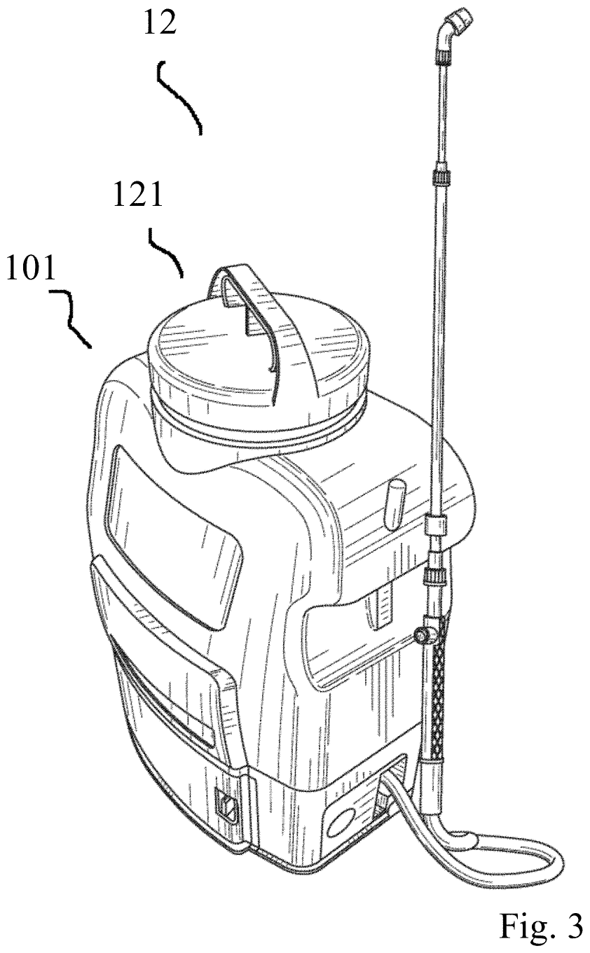

FIG. 3 shows a further embodiment of the present invention. FIG. 3 provides electrostatic sprayer 12 with a tank 101 and a tank lid 121 that includes a handle. According to the embodiment shown in FIG. 3, the tank lid 121 is centered on tank 101 allowing for the user to carry electrostatic sprayer 12 in a balanced fashion.

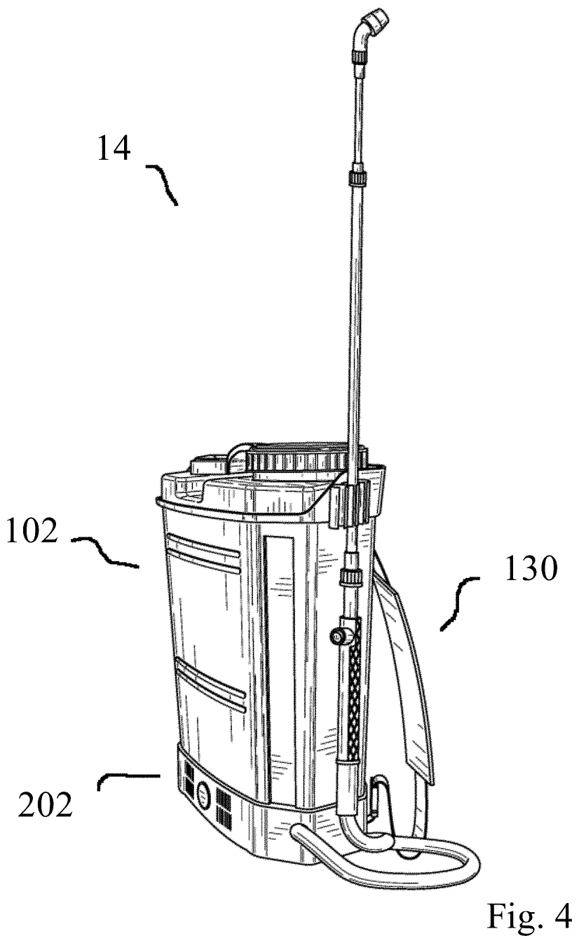

FIG. 4 shows yet a further embodiment of an electrostatic sprayer 14 with a longer and narrower solution tank 102 and a narrow base 202. Electrostatic sprayer 14 also shows backpack straps 130. While a variety of embodiments of the present invention have been shown, it is clear that many configurations may be constructed without diverting from the intent and function of the present invention. For simplicity, the further description of the present invention will use various views of electrostatic sprayer 10.

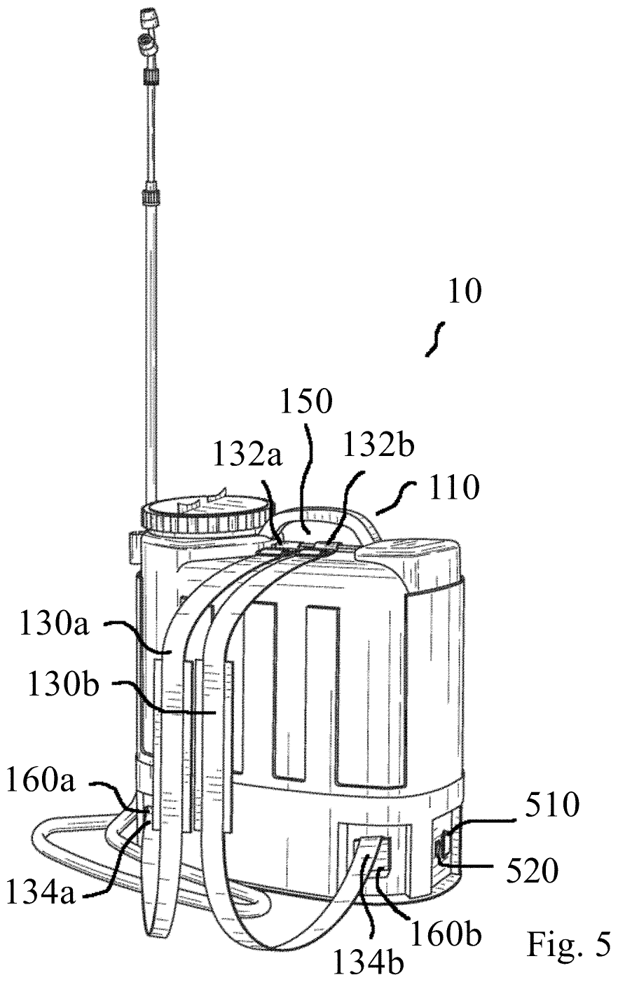

FIG. 5 provides a reverse perspective view of electrostatic sprayer 10. In this view, backpack straps 130a and 130b are shown. Each backpack strap 130a and 130b have a first end 132a and 132b and a second end 134a and 134b. The first ends 132a and 132b of each backpack strap 130a and 130b are centrally attached to the top of the solution tank 100 at tank backpack attachment point 150. The second end 134a and 134 b of each backpack strap 130a and 130b are attached to base backpack attachment points 160a and 160b. The backpack straps 130a and 130b are used for carrying the electrostatic sprayer 10 on the back of the user while in use or for transport or alternatively as carry handles.

Further embodiments may also include a hip strap or a chest strap for more securely attaching the electrostatic sprayer 10 to the user. A hip strap helps to balance the weight between the hips and shoulders of the user, while the chest strap keeps the backpack straps 130a and 130b from separating and falling off the shoulders of the user.

Charging port 510 and electrostatic power switch 520 are also visible in FIG. 5 and will be described in more detail with the electrical components.

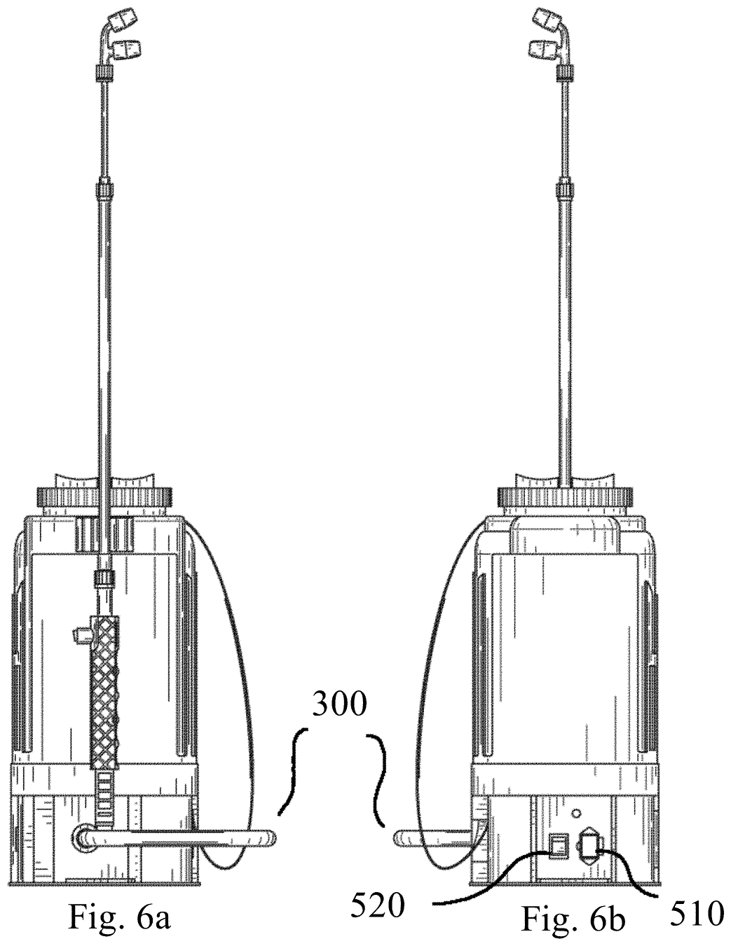

FIG. 6a provides a right-side view of the electrostatic sprayer 10 and additional detail of the supply hose 300 exiting from the base 200. FIG. 6b provides a left-side view of the electrostatic sprayer 10 and a further view of the charging port 510 and the electrostatic switch 520.

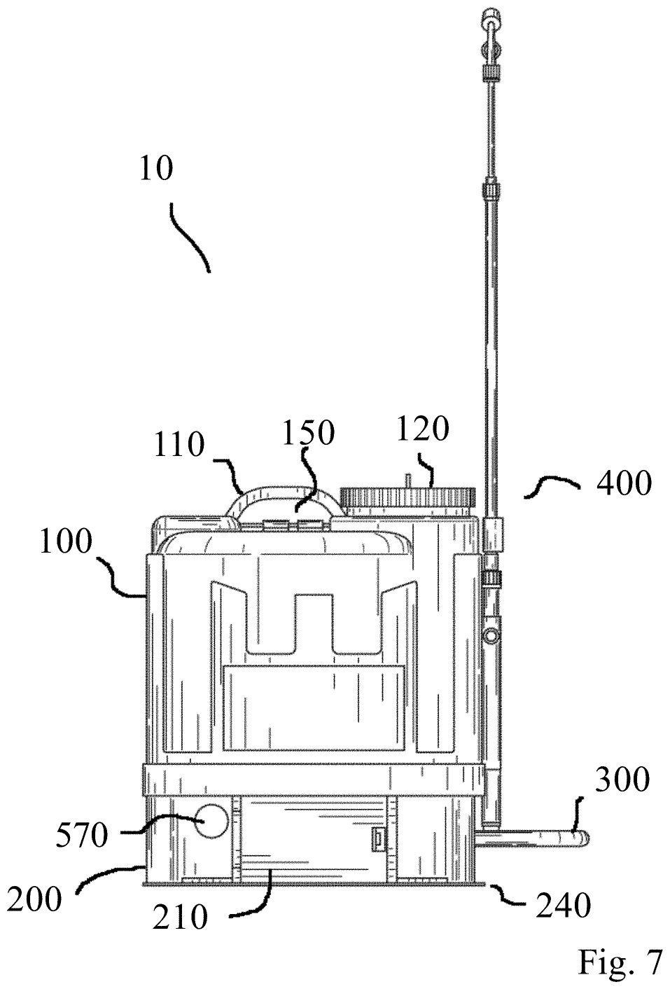

FIG. 7 shows a front view of the electrostatic sprayer 10 according to an embodiment of the present invention. FIG. 7 provides a further view of the solution tank 100, the handle 110, the tank lid 120, the backpack attachment point 150, the base 200, the access door 210, the supply hose 300, and spray wand 400. FIG. 7 also provides a view of a voltage meter 570 and a removable base plate 240. Voltage meter 570 displays the current power supply voltage providing the user with the current charge level of the electrostatic sprayer 10. With the current charge level the user can discern if the electrostatic sprayer 10 is ready for use or if the power supply should be recharged.

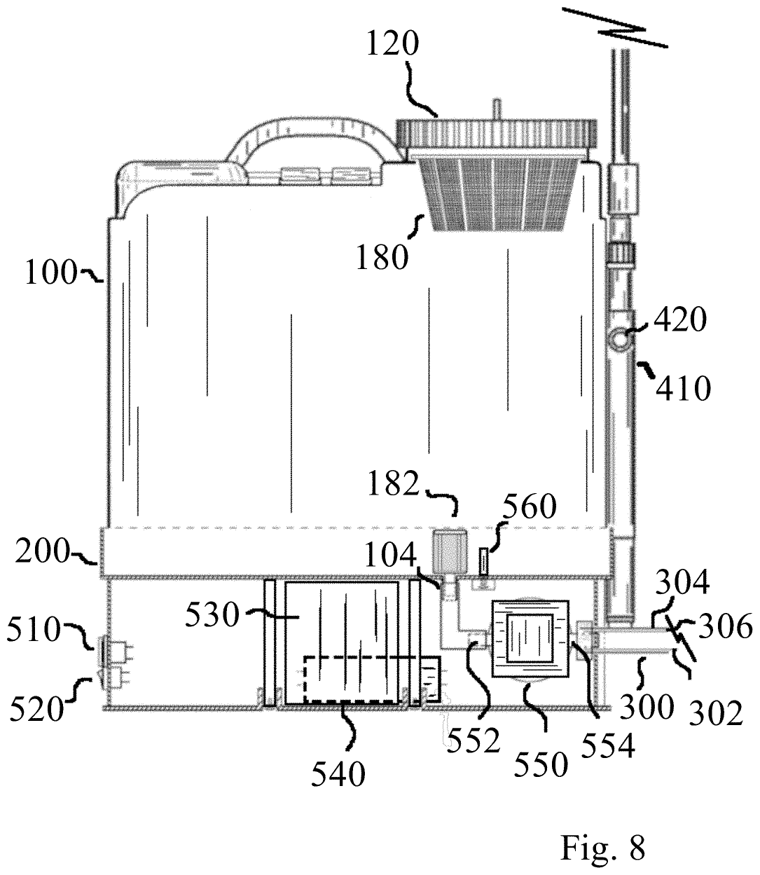

FIG. 8 shows a bisected view of the electrostatic sprayer providing an internal view of the solution tank 100 and the base 200. FIG. 8 provides a view of a filtration system within solution tank 100. The filtration system includes a fill filter 180 located within the fill opening underneath the tank lid 120 and filters the treatment solution as it is poured into the solution tank 100 prior to use of the electrostatic sprayer 10. The filtration system further includes a discharge filter 182 where the fluid is drawn out of the solution tank 100 and provides further filtration for smaller particulates that may have entered into solution tank 100.

FIG. 8 also shows the electrical components of the electrostatic sprayer 10, including the charging port 510, the electrostatic power switch 520, a battery 530, an electrostatic generator 540, a solution pump 550, a tank electrode 560, and the voltage meter 570.

When the wand switch 420 is in the on position, the solution pump 550 pumps the treatment solution from the solution tank 100 through an exit port 104 in the solution tank into the solution pump 550 through an inlet port 552. According to one embodiment of the present invention, inlet port 552 interconnects with the exit port 104 with flexible tubing. It will be clear to one skilled in the art that a variety of materials may be used for interconnected the inlet port 552 with the exit port 104, including pvc piping, rubber or silicon tubing, or others.

Solution pump 550 pumps the treatment solution out an exit port 554 to the supply hose 300. Exit port 554 is interconnected with the supply hose 300. Supply hose 300 further comprises internal tubing 302 and external conduit 304. A wire passage space 306 is created between internal tubing 302 and external conduit 304 and allows for electrical wires to be passed from the base 200 to the wand handle 410 for the wand switch 420 and a grounding plate 430.

It is also noted that according to the embodiment shown in FIG. 8 that when the wand switch 420 Is in the on position the supply pump 550 is turned on, but the electrostatic generator 540 is not turned on. By separating the power supply switches the electrostatic sprayer 10 may be used without the electrostatic generator 540 in situations where an electrostatically charged treatment solution could damage items within the treatment area.

The power supply 530 shown in FIG. 8 is shown filling the power supply bay. However, in further embodiments of the present invention, moveable walls (not shown) may be inserted to create a smaller power supply bay for using smaller power supplies 530. Power supplies of various sizes and weights, but having appropriate power specifications may be useful in various situations. The moveable walls allow for use of different power supply sizes yet ensure that the power supply 530 remains securely held within the power supply bay.

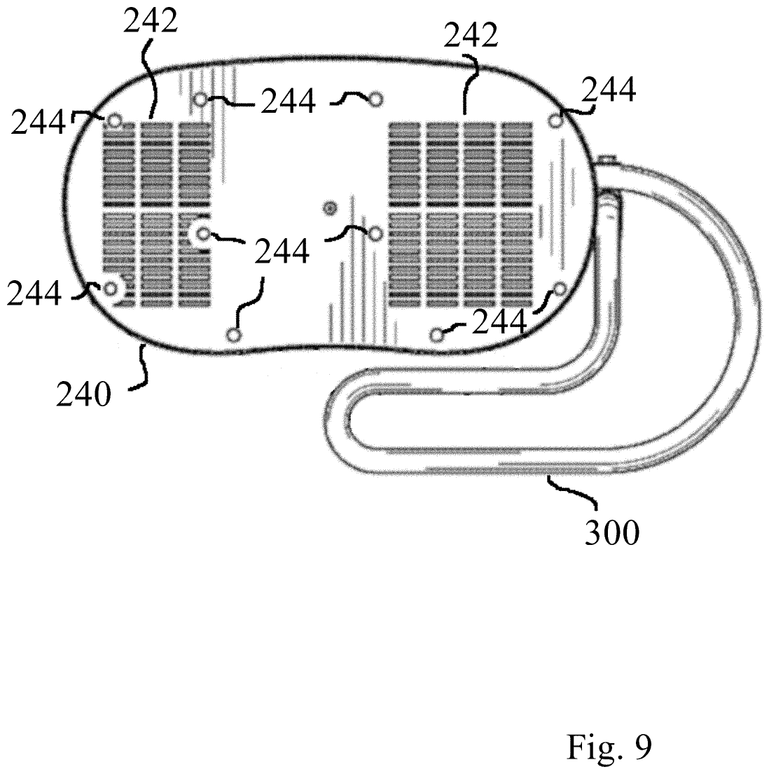

FIG. 9 provides a bottom view of the electrostatic sprayer 10 with a detailed view of the base plate 240 with the supply hose 300 for reference. According to this embodiment the base plate is removeably attached to the base 200 with screws through screw holes 244. The base plate 240 further includes vent openings 242 to provide airflow through the base 200 for ventilating and cooling the components within the base 200.

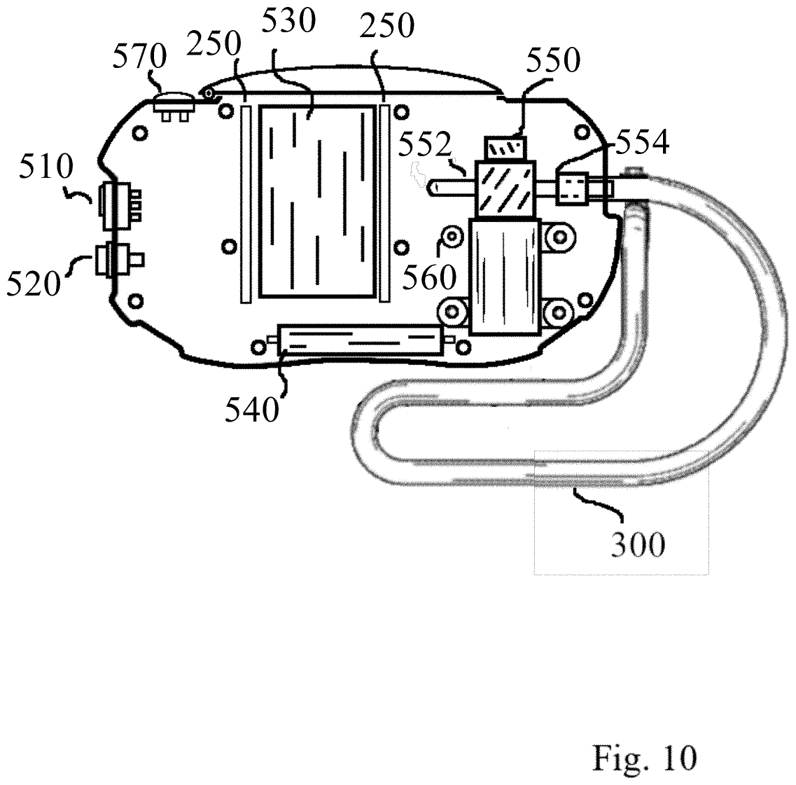

FIG. 10 shows a bottom view of the electrostatic sprayer 10 with the base plate 240 removed. With the base plate 240 removed, FIG. 10 provides a view of the components as they are situated inside the base 200 according to an embodiment of the present invention, including the charging port 510, the power switch 520, the battery 530, the electrostatic generator 540, solution pump 550, tank electrode 560, and voltage meter 570. FIG. 10 further provides a view of the moveable walls 250 holding the power supply 530 securely within the power supply bay of the base 200. The moveable walls 250 are located within base 200 by sliding the moveable walls 250 into slots within the base 200 and the base plate 240.

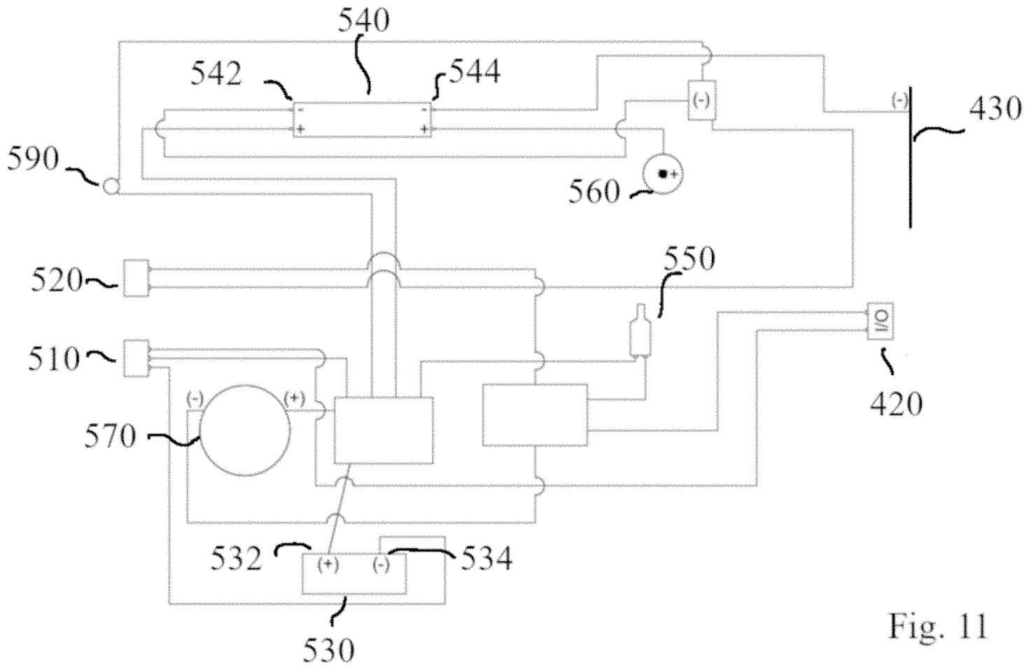

FIG. 11 shows a wiring diagram for the electrical connections and component interactions according to an embodiment of the present invention. Beginning with the power supply 530, which supplies power to all electrical components within the electrostatic sprayer 10. The power supply 530 provides a positive terminal 532 and a negative terminal 534. The positive terminal 532 is connected directly to positive terminals of the charging port 510, the electrostatic generator 540, the fluid pump 550, and the voltage meter 570.

The negative terminal 534 connects to the negative terminal of the charging port 510 providing a negative electrical connection for a battery charger as well as a pass-through negative connection that electrically connects the negative terminal 534 to the master wand switch 420. The master wand switch 420 provides an on and off position wherein the master wand switch 420 provides electrical continuity of the negative connection when the master wand switch 420 is in the on position. When the master wand switch 420 is in the off position, the master wand switch 420 shorts the electrical continuity of the negative connection. In the on position the master wand switch 420 negative continuity is provided to the solution pump 550, the volt meter 570, and to an electrostatic on/off switch 520. In turn, the electrostatic on/off switch 520 further provides electrical continuity when it is in the on position and shorts continuity when in the off position.

As shown in FIG. 11, the electrostatic generator 540 further includes a low voltage side 542 with a positive and negative terminal and a high voltage side 544 with a positive and negative terminal. When the electrostatic on/off switch 520 and the master wand switch 420 are both in the on position there is negative continuity between the negative terminal 534 and the negative terminal on the low voltage side 542 of the electrostatic generator 540; thus completing the circuit and powering the electrostatic generator 540.

The positive terminal 532 is connected directly to the positive terminal of the low voltage side 542 of the electrostatic generator 540. Accordingly, when there is negative continuity from the negative terminal 534 and the negative terminal on the low voltage side 542 of the electrostatic generator 540 the electrostatic generator 540 is powered on.

The electrostatic on/off switch 520 allows a user to turn the electrostatic generator 540 on or off while the solution pump 550 will run with or without an electrostatic charge being generated in the treatment solution. Such a configuration allows a user to turn off the electrostatic generator when treating areas or items with sensitivity to an electrostatic charge, such as micro-electronic components or areas with flammable vapors, for example.

As further shown in FIG. 11, when the electrostatic generator 540 is powered on it supplies a positive charge through the positive terminal on the high voltage side 544 to the tank electrode 560; thus providing a positive charge to the treatment solution inside the solution tank 100. When powered on the negative terminal on the high voltage side 544 provides a negative charge to the grounding strip 430 in the wand handle 410. According to further embodiments, tank electrode 560 is preferably made of brass to limit or reduce corrosion from regular immersion in the treatment solution.

In a further embodiment, an electrostatic light 590 is interconnected with the battery positive terminal 532 and the electrostatic on/off switch 520. When the circuit is completed and the electrostatic generator 540 is powered on, the electrostatic light 590 is also powered on providing a visual indicator that the electrostatic generator 540 is powered on.

While a user is in contact with the grounding strip 430 in the spray wand 400 the present invention provides a grounding path between the electrostatic generator 540 and earth via the user. Grounding the electrostatic generator 540 with earth provides an enhanced electrical attraction between the treatment solution and the objects being sprayed; thus, generating a more efficient coverage of the sprayed items.

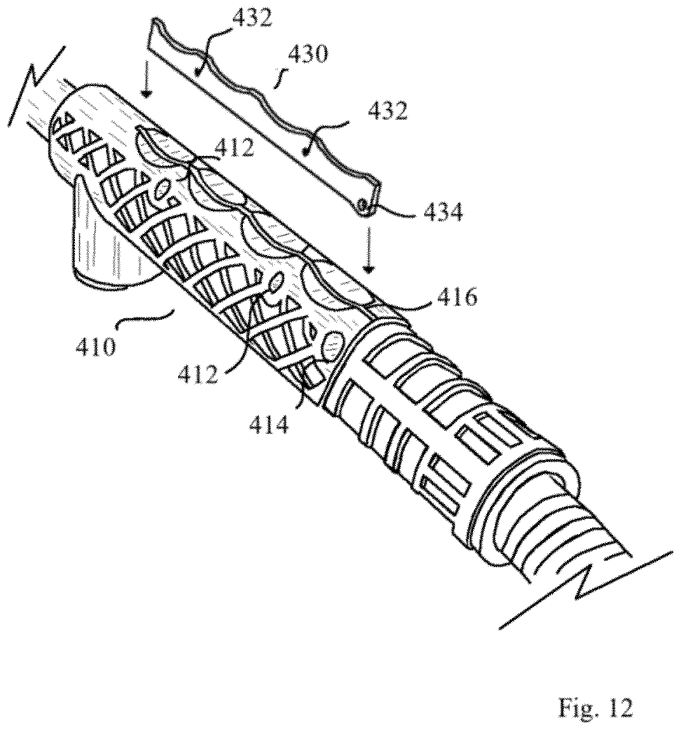

FIG. 12 shows a detailed view of the spray wand handle 410. Spray wand handle 410 further comprises a handle grounding slot 416, one or more attachment openings 412, and a grounding attachment opening 414. Grounding strip 430, as shown, further comprises grounding strip attachment openings 432 and grounding opening 434.

In use, grounding strip 430 is inserted into the handle grounding slot 416 wherein the attachment openings 412 of the spray wand handle 410 line up with the grounding strip attachment openings 432. The grounding strip 430 is then held in place with screws fastened through the attachment openings 412 and the grounding strip attachment openings 432. Similarly, grounding attachment opening 414 and grounding opening 434 line up to allow a screw to pass through and attach the negative wire attached to the high negative contact on the high voltage end of the electrostatic generator 540.

It will be clear to those skilled in the art that there are a variety of ways to attach the grounding strip 430 to the spray wand handle 410, including but not limited to a simple compression fit, using rivets, compression posts, or any combination thereof, in place of the screws. Similarly, it will be clear to those skilled in the art that the grounding opening 434 may attach to the negative wire with solder, a rivet, or other connecting device.

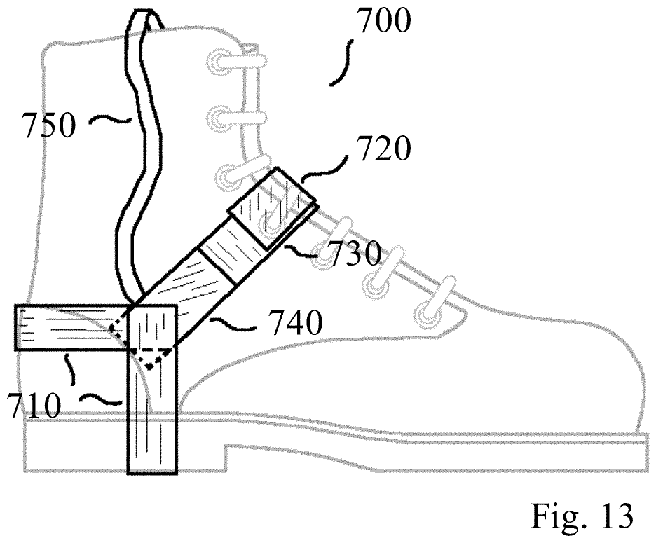

Turning to FIG. 13, a grounding strap 700 is shown for use with various embodiments of electrostatic sprayer 10. As previously discussed, the grounding strip 430 in the spray wand handle 410 provides a grounding path from the electrostatic generator 540 through the user to Earth for enhancing the electrostatic attraction between the positively charged treatment solution and the surface being treated.

According to further embodiments of the present invention, the grounding strap 700 may be used to further enhance the grounding path from the user to Earth. The grounding strap 700 provides a lower resistance path between the grounding strip 430 and Earth by bypassing some of the user's clothing and protective gear that may insulate the path between the grounding strip 700 and Earth. Grounding strap 700 includes a foot strap 710 for attaching to the sole of the user's shoe. To secure the foot strap 710 to the user's shoe a first strap 720 and a second strap 730 removeably interconnect and allow the user to adjust the firmness of the foot strap 710. An elastic strap 740 is also connected between the second strap 730 and the foot strap 710 to provide some flexibility in the connection around the user's foot.

As shown in FIG. 13, grounding strap 700 further includes a grounding lace 750. A first end of the grounding lace 750 is connected to the foot strap 710. The second end of the grounding lace 750 may be placed in the user's shoe or sock. Accordingly, the grounding path through the user is effectively enhanced by bringing the grounding path to Earth on the outside of the user's shoe, which limits any insulating effect a user's footwear may provide.

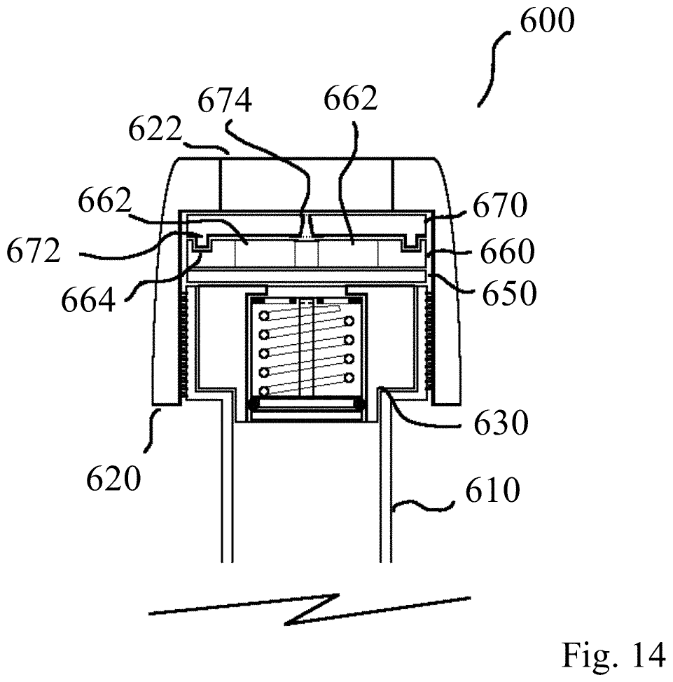

FIG. 14 shows a bisected view of a spray nozzle 600 according to an embodiment of the present invention. As discussed previously, various embodiments of the present inventions may include one or more spray nozzles 600. Each spray nozzle 600 is attached to a nozzle base 610 extending from the spray wand 400. The internal components of the spray nozzle 600 are held in place with a nozzle cap 620 removeably connected with the nozzle base. As shown in FIG. 14, the nozzle base 610 further includes external threading and the nozzle cap 620 further includes internal threading allowing the cap to be securely connected by screwing the two together.

When connected, the nozzle base 610 and the nozzle cap 620 house a backflow valve 630, a washer 650, a first metering disk 660, and a second metering disk 670 stacked together as shown to meter the flow of treatment solution and stopping leaking and backflow of treatment solution when the electrostatic sprayer 10 is turned off.

In use, the treatment solution is pressurized and fed to the spray wand 400. At each valve the pressurized solution flows through the backflow valve 630, through the washer 650 and into the first metering disk 660. The first metering disk includes one or more metering openings 662 for allowing pressurized treatment fluid to pass through. The first metering disk 660 further includes a groove 664 concentrically located on the top face of the first metering disk 660. The groove 664 of the first metering disk 660 interconnects with a protruding ring 672 concentrically located on the bottom face of the second metering disk. 670.

The interconnection of the groove 664 and the ring 672 ensures proper alignment of the first metering disk 660 and the second metering disk 670 further providing alignment of the metering openings 662 with an atomization opening 674 centrally located in the second metering disk 670. By changing size of the metering openings 662 and atomization opening 674 the size of the atomized droplets of treatment solution may be adjusted for differing applications. In a preferred embodiment, the first metering disk 660 and the second metering disk 670 atomize the treatment solution to droplets of approximately 40 microns.

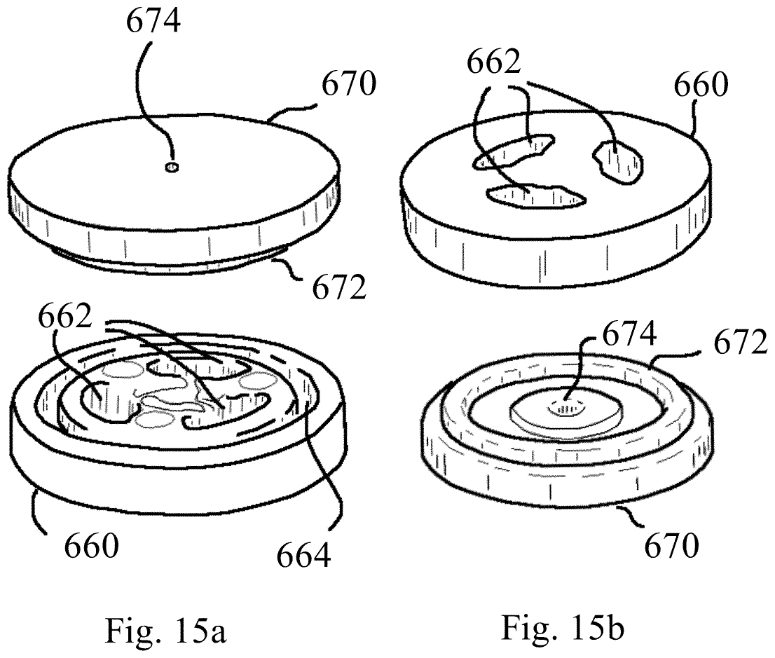

FIGS. 15a and 15b provide detailed views of the first metering disk 660 and second metering disk 670. FIG. 15a specifically shows a perspective view of the top sides of the first and second metering disks 660 and 670. The first metering disk 660 as shown in FIG. 15a shows the groove 664 and the one or more metering openings. The second metering disk 670 shows the atomization opening 674 centrally located on the top of the second metering disk 670 and the ring 672.

FIG. 15b shows a perspective view of the bottom sides of the first and second metering disks 660 and 670. The one or more metering openings 662 an be seen on the bottom of the first metering disk 660. The atomizing opening 674 and ring 672 are also seen on the bottom side of the second metering disk 670.

In use the pressurized solution passes through the metering openings 662 of the first metering disk 669 and into the atomization opening 674 of the second metering disk 670. It will be understood by one skilled in the art that the size of the openings and the shapes of the matching faces can be modified to provide different flow rates to match the specified use a specific electrostatic sprayer according to various embodiments of the present invention.

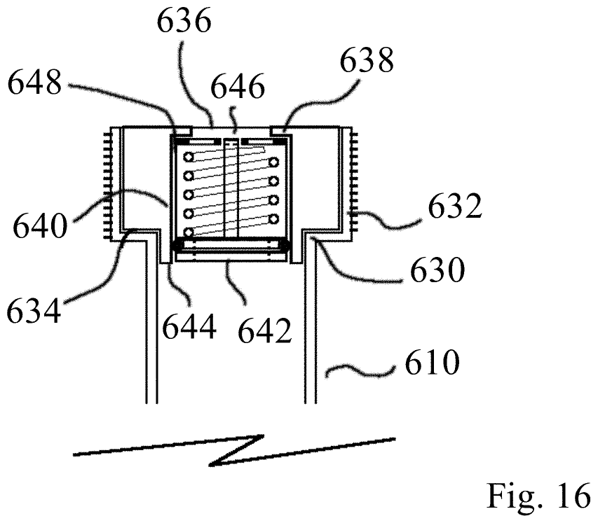

FIG. 16 provides a bisected view of the backflow valve 630 within the nozzle base 610. According to the embodiment shown in FIG. 16, the backflow valve 630 further comprises a brass ferrule 632 with an external ferrule shoulder 634 formed at the bottom end of the ferrule 632 for fitting within the nozzle base 610. The ferrule 632 further includes a central opening 636 passing from the top end to the bottom end of the ferrule 632. The central opening 636 forms an internal shoulder 638 at the top end of the ferrule 632. In a preferred embodiment of the present invention, the ferrule 632 is made of brass. It will be apparent to one skilled in the art that the ferrule 632 may be made of a variety of materials, including other metals, plastics, or composites.

As further shown in FIG. 16 a valve body 640 is disposed within the central opening 636. The valve body 640 further includes an o-ring groove 642 near the base of the valve body 640. A valve body o-ring 644 fits within the o-ring groove 642 to provide a seal between the valve body 640 and the ferrule 632. A valve plunger 646 is placed within the valve body 640 and a valve spring 648 keeps the valve plunger 646 moveably closed when there is no pressurized solution coming through the nozzle base 610 pushing against the plunger 646.

When the wand switch 420 is placed in the on position the solution pump 550 is turned on and the treatment solution is drawn from the tank 100 and pumped to the supply hose 300 and the spray wand 400, wherein the valve plunger 646 is pushed open allowing the treatment solution to flow through the spray nozzle 600 and is metered by the first and second metering disks 660 and 670. When pressurization of the solution treatment is stopped the valve plunger 646 is pushed closed by the valve spring 648. When the valve plunger 646 is in the closed position the treatment solution will not be able to leak out of the spray nozzle 600, well as prevent a siphon from being created by the flow of treatment solution out of a nozzle.

In summary, the electrostatic sprayer as disclosed above provides examples that are illustrative of some of the applications of the principles of the present subject matter. Thus it is contemplated that the electrostatic sprayer may be constructed for many different applications and alterations may be made wherein an electrostatic sprayer may include many of the above-mentioned advantages. Further additions or alterations may be made to the sprayer and may be made without departing from the spirit and scope of the present disclosure. Furthermore, those skilled in the art will understand that a number of variations, including those described, may be made to the materials, electronics and other elements, and the processes for making the electrostatic sprayer, all without departing from the scope of the invention, which is described above and defined by the appended claims.

What is claimed is:

1. An electrostatic sprayer comprising:
a solution tank;
a base;
a supply hose;
a spray wand having a wand handle with a grounding strip; and
a grounding strap.

2. The electrostatic sprayer of claim 1 further comprising:
a power supply;
a tank electrode disposed within the solution tank;
an electrostatic generator electrically connected to the grounding strip and the tank electrode; and
a solution pump.

3. The electrostatic sprayer of claim 2 further comprising a switch in the wand handle electrically connected with the solution pump for providing electrical continuity for turning the solution pump on or off.

4. The electrostatic sprayer of claim 2 further comprising an electrostatic generator switch interconnected with the electrostatic generator to provide electrical continuity for turning the electrostatic generator on or off.

5. The electrostatic sprayer of claim 2 wherein the electrostatic generator further comprises a high voltage end with a positive terminal and a negative terminal, wherein the electrostatic generator high voltage positive terminal is electrically connected to a tank electrode and the electrostatic generator high voltage negative terminal is electrically connected to the grounding strip.

6. An electrostatic sprayer comprising:
a solution tank with a fill opening and an exit opening;
an inlet filter removeably disposed within the fill opening;
an exit filter removeably disposed within the exit opening;
a base;
a supply hose;
a spray wand having a wand handle with a grounding strip;
a tank electrode; and
an electrostatic generator electrically connected to the grounding strip and the tank electrode, wherein the electrostatic generator charges treatment solution within the tank and the grounding strip passes the opposite charge to Earth to create an electrostatic attraction between the treatment solution and items being treated.

7. The electrostatic sprayer of claim 6 further comprising a grounding strap to reduce the resistance of the electrical path between the grounding strip and Earth.

8. An electrostatic sprayer comprising:
a solution tank;
a base;
a supply hose; and
a spray wand having a wand handle with a grounding strip and one or more spray nozzles.

9. The electrostatic sprayer of claim 8, the one or more spray nozzles further comprising a spray nozzle base, a nozzle cap, a backflow valve, a nozzle gasket, a first metering disk, and a second metering disk.

10. The electrostatic sprayer of claim 9, the first metering disk further comprising a first side, a second side, and one or more openings passing through the first metering disk from the first side to the second side providing a path for pressurized treatment solution to pass through the first metering disk.
wherein the second side further comprises a groove concentrically formed near the outer edge of the first metering disk for interconnecting with the second metering disk.

11. The electrostatic sprayer of claim 10, the second metering disk further comprising a first side, a second side, and an atomizing opening passing through the center of the second metering disk from the first side to the second side providing a path for pressurized treatment solution to pass through the first metering disk.
wherein the second side further comprises a ring concentrically formed near the outer edge of the second metering disk for interconnecting with the groove of the first metering disk.

12. The electrostatic sprayer of claim 9, the backflow valve further comprising a ferrule and a valve body disposed within the ferrule.

13. The electrostatic sprayer of claim 12, wherein the valve body further comprises
an o-ring groove,
a valve plunger, and
a valve spring,
wherein the valve plunger is pressed closed by the valve spring when no pressurized treatment solution is in the spray wand.

14. The electrostatic sprayer of claim 13, wherein the backflow valve further comprises an o-ring located within the o-ring groove providing a sealed fit between the valve body and the ferrule.
